(19) United States
(12) Patent Application Publication
(10) Pub. No.: US 2014/0358204 A1
(43) Pub. Date: Dec. 4, 2014

(54) METHOD AND APPARATUS FOR CONTROLLING MENOPAUSAL HOT FLASHES

(71) Applicant: Robert G. Dickie, King City (CA)

(72) Inventor: Robert G. Dickie, King City (CA)

(21) Appl. No.: 13/906,998

(22) Filed: May 31, 2013

(51) Int. Cl. A61F 7/00

(52) U.S. Cl. CPC A61F 7/007; A61F 2007/0011; A61F 2007/0075; A61F 2007/0087
USPC 607/109; 607/96

(57) ABSTRACT

A device and method for non-invasive treatment of menopausal hot flashes. The device includes a hand-held assembly sized to be held in the palm of a user's hand; a cooling plate within the assembly and a cradle which docks with the assembly. The cradle plugs into an AC power source and includes a Peltier-effect element for cooling a contact plate. The contact plate in the cradle abuts the cooling plate in the docked assembly and cools the same, keeping it at the lowered temperature and ready for immediate use. The cradle includes a heat sink for dissipating heat generated by the Peltier-effect element. The assembly is undocked from the cradle. The cooled plate is placed against the skin on the back of the neck and is moved back and forth to stimulate cold thermoreceptors in the skin, thereby counteracting the false trigger which caused the hot flash.

### BACKGROUND OF THE INVENTION

1. Technical Field

This invention generally relates to therapeutic devices. More particularly, the invention relates to a device that is useful for controlling or stopping a menopausal hot flash. Specifically, the device includes at least one plate that is cooled using a Peltier-effect element and the cooled plate is moved back and forth across the skin on the back of the neck to stimulate cold thermoreceptors in the skin and thereby counteract the false triggering of the hypothalamus which causes a hot flash.

2. Background Information

Mammals are warm blooded creatures that are able to thrive in a wide range of environments for a number of reasons. One of these reasons is that that their bodies are provided with complex thermoregulatory systems that enable them to monitor the ambient temperature, i.e., the temperature of their surroundings, and to adjust various bodily functions to keep their internal core temperature substantially constant. The core temperature is vital to the survival of the mammal in that if it becomes too high they will die and if it drops too low they will die. If the ambient temperature increases, the mammal's body will tend to absorb some of this surrounding heat and their core temperature will tend to rise in response. Similarly, if the ambient temperature drops, the body will naturally radiate heat into the environment thereby causing a drop in the body's core temperature. The thermoregulatory system will adjust body functions so as to lose heat, prevent heat loss or even generate heat in order to keep the core temperature more or less constant.

In humans, who are mammals, one of the most important parts of this thermal monitoring and regulation system is the skin. Skin includes both hot and cold temperature receptors (thermoreceptors) that detect the ambient temperature and then fire a signal to the hypothalamus. The hypothalamus takes action in response to this data and changes various bodily functions to regulate the core temperature. These bodily functions controlled by the hypothalamus include, but are not limited to, the body's heart rate, the rate of breathing, the extent of blood flow to the extremities, and rates of sweating and shivering. If, for example, the thermoreceptors in the skin indicate that the environment is too hot and that the core temperature is therefore likely to rise, the heart rate will be increased and blood flow to the capillaries that are just beneath the skin's surface will be increased. Both of these actions bring warmed blood closer to the skin very rapidly so that heat can radiate from the blood to the environment. Additionally, the sweat glands are stimulated and liquid is released onto the skin's surface. Evaporation of this liquid utilizing heat from the blood close to the skin's surface causes the temperature of the skin, the blood and therefore the body's core to be lowered. If, on the other hand, the receptors detect that the environment is too cold and that the core temperature is therefore in jeopardy of falling too low, the thermoregulatory system will slow the heart rate and will decrease blood flow to the body's extremities which causes capillaries close to the skin's surface to close. This slows down the rate at which heat is radiated into the environment from the body. Furthermore, hair follicles on the skin's surface are stimulated to vibrate, a condition typically referred to as shivering. This generates heat which flows into the blood. These actions help to keep the body's core temperature in a safe range.

As any menopausal woman knows, one of the most unpleasant symptoms of this time of life is the disruption in the body's complex thermoregulatory system. There is periodically a false triggering of the thermoregulatory system which affects or is internal to the hypothalamus. This false trigger causes the hypothalamus to determine that the body's core temperature is rising rapidly and is getting dangerously high. The hypothalamus responds by taking the action that is typically required to lower the core temperature. This response includes vascular dilation and an increase in heart rate to get blood more rapidly to the skin for cooling. These actions cause the woman to feel flushed, out of breath and extremely hot as the blood is rushed to the skin. In other words, the woman has a hot flash. The result of this hypothalamic action is that the body's core temperature drops by as much as two to three degrees Celsius. Once the hot flash is over, the woman may experience chills for an hour or more as her body attempts to build back up to a safer core temperature range. While hot flashes cause the body temperature to rise rapidly for one or two minutes and are then gone, these extreme temperature swings can leave the woman drained and emotionally distraught. While this condition is hormonally cradled and is therefore extremely difficult to avoid or correct, it is desirable that at least the symptoms thereof be addressed in some manner that will bring the menopausal woman relief and leave her with a greater sense of being in control of her own body.

The present inventor has recognized that the body's own thermoregulatory system may be utilized to aid in reducing the menopausal symptoms known as hot flashes. Furthermore, the inventor has recognized that the skin plays a large role in assisting to keep the core temperature of the body more or less constant. Even further, the inventor has recognized that the skin at the back of the neck has a very high density of thermoreceptors and that it is a key area of the body that the hypothalamus monitors in determining ambient temperature.

The prior art has disclosed various devices and modalities for the treatment of other ailments that utilize neural pathways and for cooling the body. One such device and modality is disclosed in U.S. Pat. No. 5,632,769 to Saringer. Saringer's device is used in the treatment of intense localized pain and includes a mechanism for creating a spatial temperature differential in one of the device's surfaces. The temperature differential is set up in a surface area that is around 1 square centimeter in size, and the mechanism generates an intensely high temperature in a first region of this surface and an intensely low temperature in a second region of this surface. The high temperature is maintained at around 45 degrees Centigrade and the low temperature is maintained at around 0 degrees Centigrade. The temperature differential is therefore around 45 degrees Centigrade across this 1 square centimeter. This small surface area is then placed in contact with the user's skin in the immediate area experiencing pain and is kept in place for a period of 15 to 24 minutes. The sensation generated by the device is felt by the user as intense heat. The temperature differential generates a large neural signal that travels along much the same neural pathways as the pain signals would travel, and the pain signals are effectively blocked by this device.

U.S. Pat. No. 6,165,640 (Taylor et al) discloses a device for cooling or heating the body. The device is C-shaped and configured to fit around a region of the body to be heated or cooled. In particular, the C-shaped housing encircles at least 220° of the region of the body the device engages. The patent specifically discloses fitting the device around a user's head or neck. The device includes a first portion that is thicker and contains many of the functional components. Two thinner wings extend outwardly from the thicker region. It is disclosed that when the device is worn around the neck the thicker region is seated adjacent the back of the neck and then the two wings extend forwardly around the neck and toward the front thereof. The device includes a Peltier-effect element for thermal regulation, air intake and exhaust ports, and a thermally conductive heat sink region that is urged into contact with the patient's skin. The heat sink region extends along substantially the entire interior surface of the C-shaped device so that it is in contact with a substantial area of the user's skin on the back and sides of the neck. This device may be suitable for continuously cooling the user's skin and blood but would be ineffective as a menopausal hot-flash treatment device because of the constant contact with the user's skin. Maintaining constant contact with the skin will cause the thermoreceptors in the user's neck to stop firing after just a few seconds and thus any benefit derived from the device for stopping a hot flash will be at best extremely short-lived.

U.S. Pat. No. 6,229,367 (Strauss et al) is also a C-shaped member that is used to cool a region of the user's body and is specifically indicated as being positionable around the neck. The device includes a heat dissipating member which abuts the region of the user's body, a liquid-retaining material that contacts the heat dissipating member and a plenum disposed between the heat dissipating member and the liquid-retaining material. The device also includes air intake and exhaust ports and a battery-powered fan to move air through the ports and plenum. Again, the heat dissipating member remains in contact with the skin at all times and so its possible effectiveness for killing hot flashes is, at best, short lived.

US publication number 2407/0098769 (Champion) discloses a device for treating menopausal hot flashes. The device comprises a cooling device which is adhesively applied on the body of a woman experiencing a hot flash when the first symptoms of a hot flash are detected. The device is placed at a hot flash origin site as determined by the user. Two or more of these cooling devices may be placed at different sites on the body. The devices are wrapped in a sealed package which is broken open when the first symptom of a hot flash is experienced and the device is then adhered to the hot flash origin site. The device may be stored in a refrigerator or a freezer in certain instances so that it is ready to use, or the device may be manufactured from materials that produces causes evaporation from the skin (and therefore cooling), or it may cause an endothermic reaction. According to the publication, the device is placed on the skin for anywhere from about 5 minutes to about 34 minutes. An example device includes more than one cooling region so that when it is adhered to the user's skin; more than one region of the skin will be cooled. Like the previous devices, because of the constant contact of the cooling regions with the skin, the thermoreceptors in the skin will stop firing within a few seconds and thus the benefits of stopping the hot flash before it is fully developed are lost in this device.

The present inventor is also named as an inventor in US publication 2410/0225307 (now abandoned) for a hand-held device for treating menopausal hot flashes. The entire specification of this publication is incorporated herein by reference. The device includes Peltier-effect elements for cooling and is placed in abutting contact with the skin on the back of the neck. The location of the device on the skin is changed every ten to fifteen seconds to ensure the thermoreceptors in the skin keep firing and thereby aid in stopping the hot flash. One of the issues with this device is that it takes too long to cycle up, sometimes around 24 to 34 seconds. When sensing the onset of a hot flash if a user can get something very cold onto the back of the neck quickly, then the hot flash may be reversed almost completely. However, if the hot flash becomes too fully developed then while the cold will provide some measure of comfort, the hot flash itself cannot be reversed. The user then just has to ride the rest of the hot flash out. The time that the device described in the publication takes to cycle up is too long to become cold enough to stop a hot flash. It therefore simply becomes a comfort aid when applied to the skin.

There is therefore a continued need in the art for a method of aiding in the control of the menopausal symptom commonly known as a hot flash and for a device that is used for this purpose.

### SUMMARY

A device and method for non-invasive treatment of menopausal hot flashes. The device includes a hand-held assembly sized to be held in the palm of a user's hand; a cooling plate within the assembly and a cradle which docks with the assembly. The cradle plugs into an AC power source and includes a solid state cooling system, preferably a Peltier-effect element, for cooling a contact plate. The contact plate in the cradle abuts the cooling plate in the docked assembly and cools the same, keeping it at the lowered temperature and ready for immediate use. The cradle includes a heat sink for dissipating heat generated by the Peltier-effect element.

At the onset of hot flash symptoms the assembly is undocked from the cradle. The cooled plate is placed against the skin on the back of the neck and is move thereacross. Inasmuch as during a hot flash the hypothalamus has been falsely triggered into determining that the body's core temperature is rising, the device is designed to specifically counteract that false trigger. This is accomplished by the device being used to cool and thereby stimulate the cold thermoreceptors in the skin at the back of the neck. In response to being cooled, the thermoreceptors fire and emit a signal to the hypothalamus indicating that the ambient temperature is extremely cold. In response to this signal, the hypothalamus stops trying to decrease the core temperature of the body. Consequently, the woman's heart rate slows and blood is no longer rushed to the surface of the skin for cooling. Thus, the false trigger is counteracted and the hot flash effectively ceases. The cooled pad is kept in any one position on the skin for no longer than 10 to 15 seconds as this is the time it typically takes for the thermoreceptors to become saturated and stop emitting a signal to the brain. Since a hot flash typically lasts only for two to three minutes, the cold thermoreceptors need only be stimulated for a very short time in order to cause the hot flash to cease. Furthermore, since the hot flash cycle is interrupted, the body's core temperature does not drop and there is therefore no need for the body to try and raise the core temperature once again. Consequently, the subsequent adverse effects of the hot flash are also averted by using the device.

When the hot flash has been stopped using the device, the hand-held assembly is returned to the cradle where the contact plate rapidly cools it down once more. Because the cradle is substantially permanently connected to a remote power source, the contact plate is always in a cooled state and is therefore consistently available to rapidly lower the temperature of the plate in the assembly. The hand-held assembly is thus always ready for immediate use and the configuration of the device is such that the assembly is able to retain the cold for around one half hour before needing to be recharged by docking it with the cradle.

### BRIEF DESCRIPTION OF THE DRAWINGS

The device is set forth in the following description and is shown in the drawings and is particularly and distinctly pointed out and set forth in the appended claims.

FIG. 1 is a top perspective view of the device showing a first side of the hand-held assembly docked in the cradle;

FIG. 2 is a top perspective view of the device showing a second side of the hand-held assembly docked in the cradle;

FIG. 3 is an elevational view showing a first end of the device where the hand-held assembly is docked in the cradle;

FIG. 4 is a first side elevational view of the device in the docked position;

FIG. 5 is a second side elevational view of the device in the docked position;

FIG. 6 is a top view of the device;

FIG. 7 is a cross-sectional view of the device taken along line 7-7 of FIG. 6;

FIG. 8 is a bottom view of the device;

FIG. 9 is an exploded view of the device showing the component parts of the hand-held assembly and the cradle; and

FIG. 10 is an end elevational view of the device similar to FIG. 3 but showing the hand-held assembly undocked from the cradle.

### DETAILED DESCRIPTION

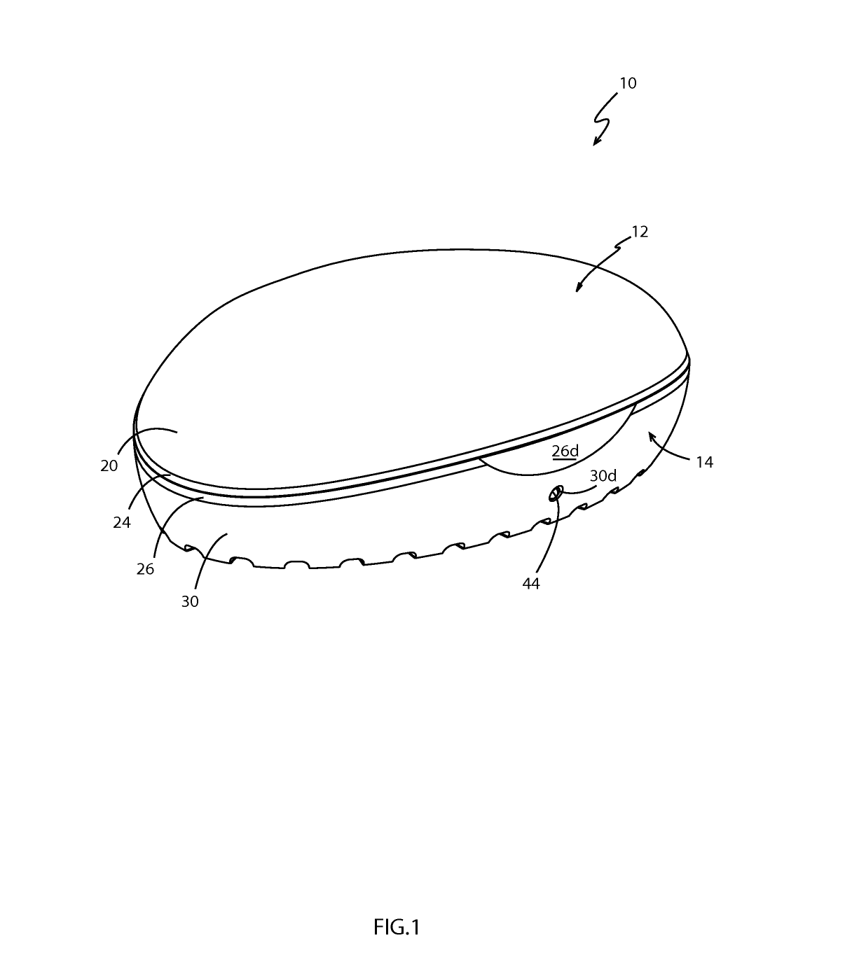

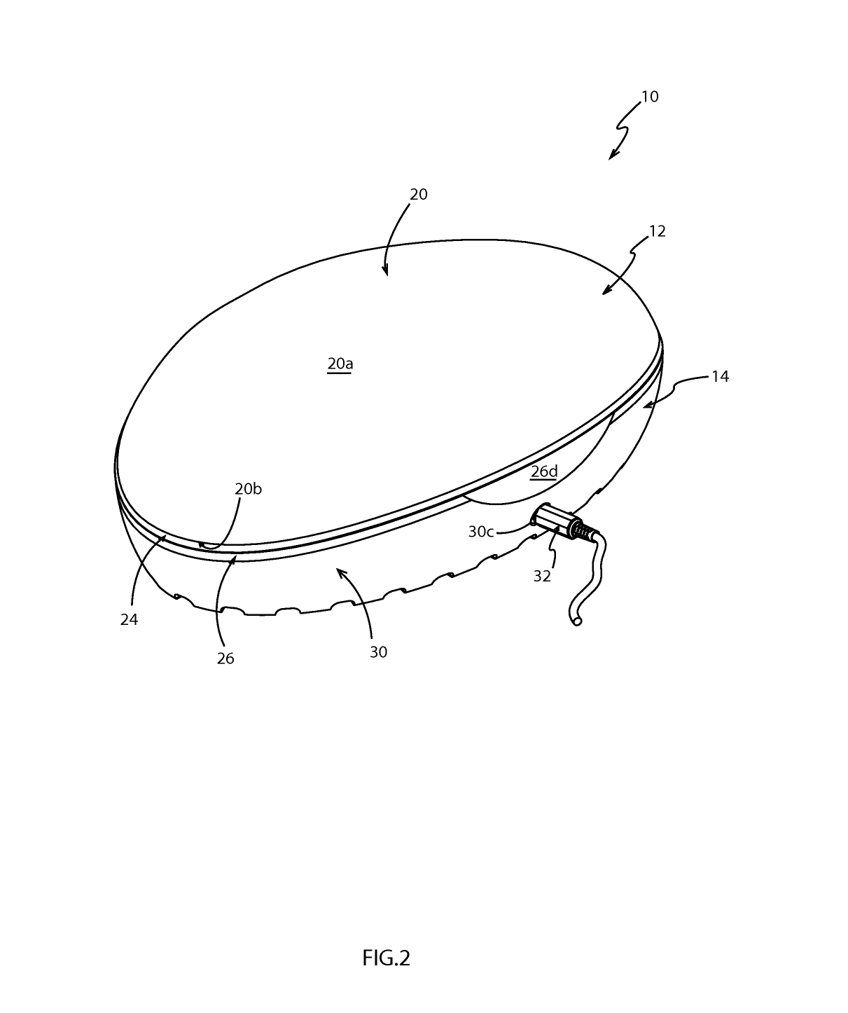

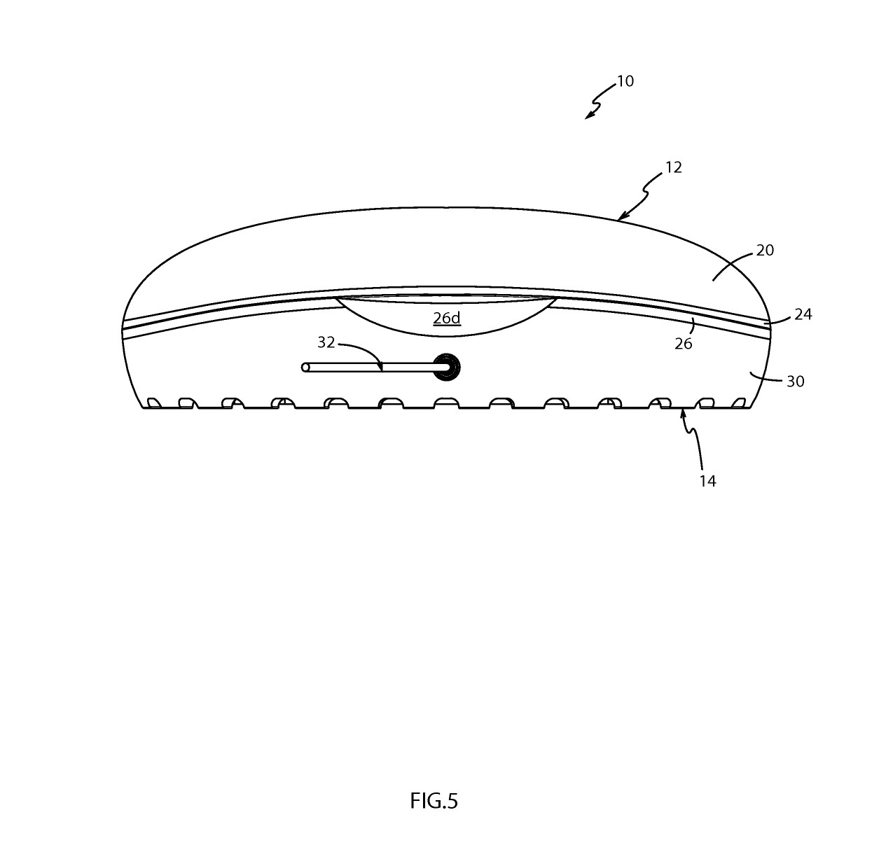

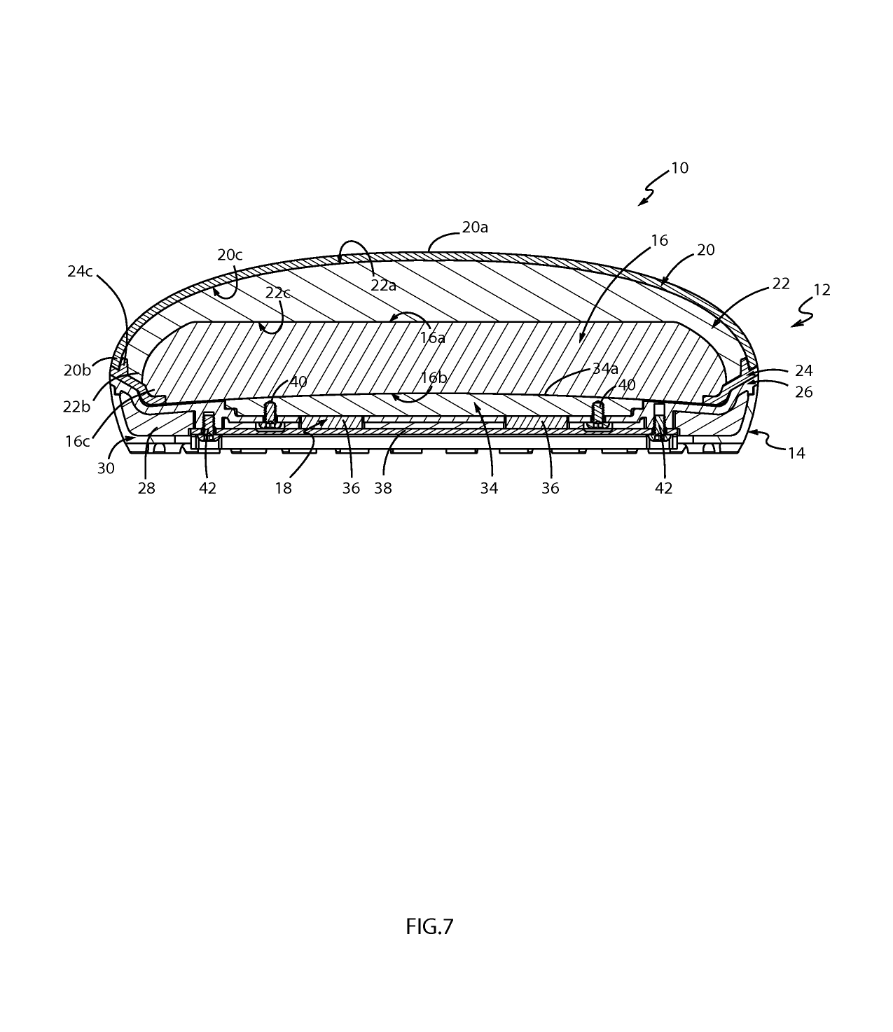

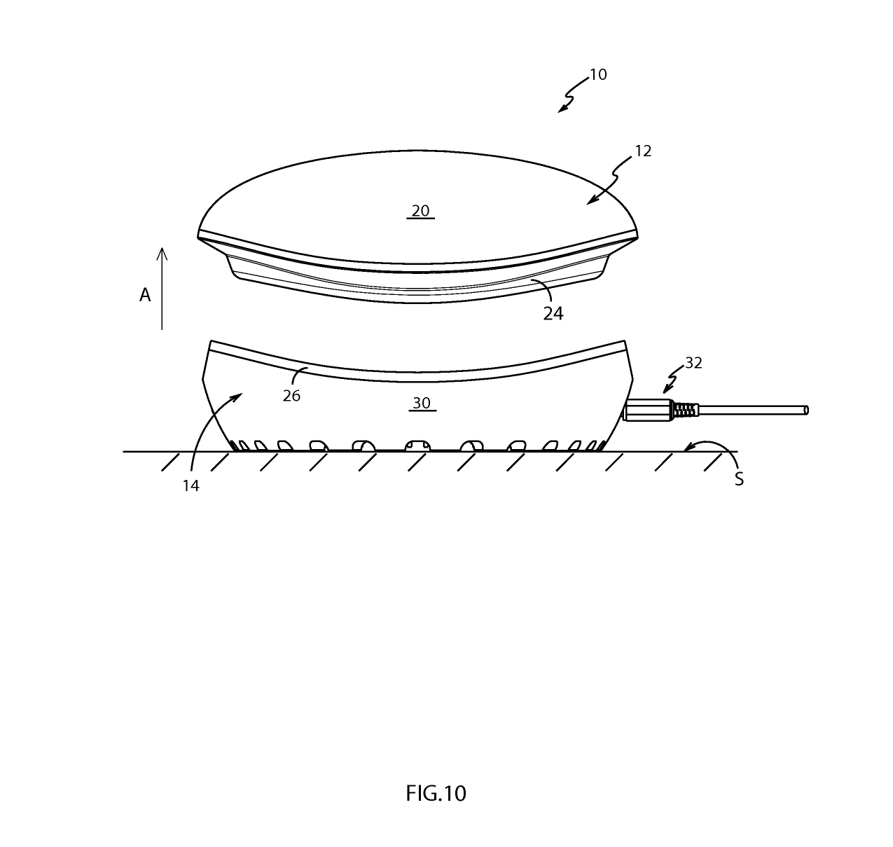

Referring to FIGS. 1-10 there is shown a device for aiding in controlling menopausal hot flashes generally indicated at 10. Device 10 comprises a hand-held assembly 12 and a cradle 14. Assembly 12 is docked with cradle 14 when not in use and is removed from cradle 14 when it is to be used to treat a hot flash. Assembly 12 includes a cooling pad 16 which engages a cooling system 18 (FIGS. 7 & 8) in cradle 14 when assembly 12 is docked in cradle 14. Cradle 14 is configured to be connected to a remote power source, such as a supply of alternating current (AC), which keeps the cooling system 18 powered up and ready to rapidly cool down the cooling pad 16. Thus, when assembly 12 is docked with cradle 14, the cooling pad 16 is maintained in a substantially constant cooled condition and is therefore always ready for use.

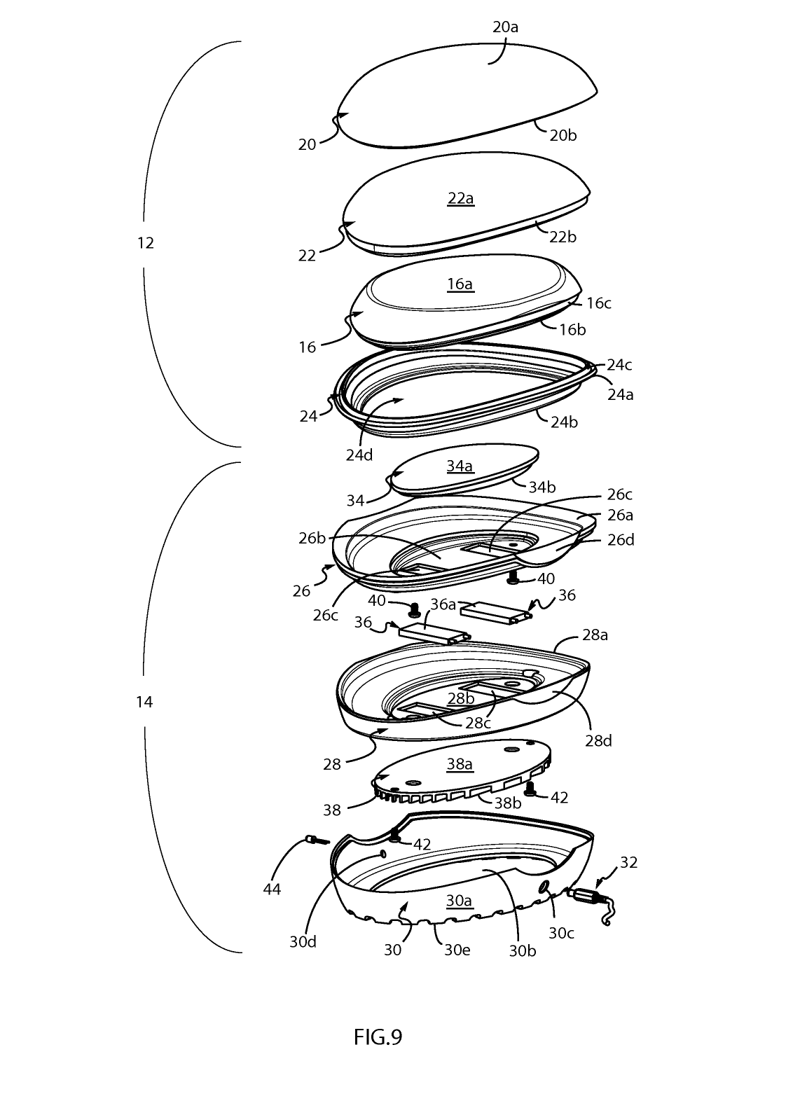

Referring particularly to FIG. 9, hand-held assembly 12 comprises a top cover 20, a first insulation layer 22, cooling pad 16, and a retainer 24. Cradle 14 includes a bottom cover 26, a second insulation layer 28, a housing 30, an AC-DC adapter 32, and a solid-state cooling system 18. Cooling system 18 includes a contact pad 34, one or more Peltier-effect elements 36, and a heat sink 38. All of these components will now be described in greater detail.

Top cover 20, retainer 24, bottom cover 26, and housing 30 are all molded from any suitable material such as Acrylonitrile Butadiene Styrene plastic (i.e., ABS plastic). First and second insulation layers 22 and 28 are fabricated from a suitable insulating material such as expanded polystyrene. Cooling pad 16, contact pad 34, and heat sink 38 are all fabricated from a suitable thermally-conductive material such as die-cast aluminum.

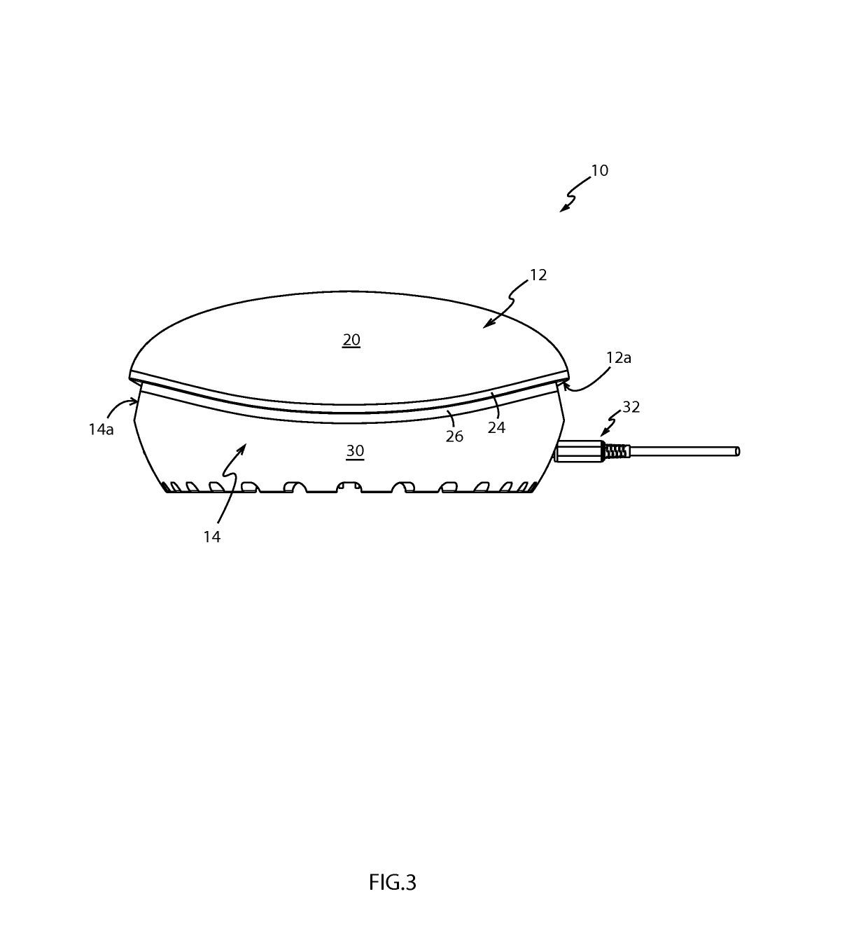

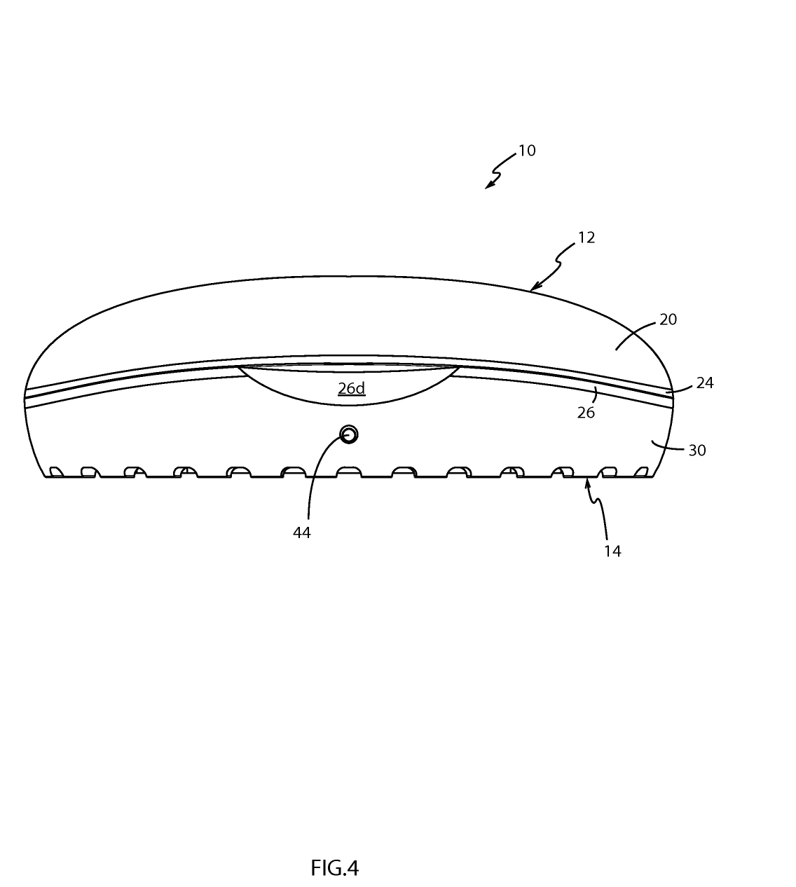

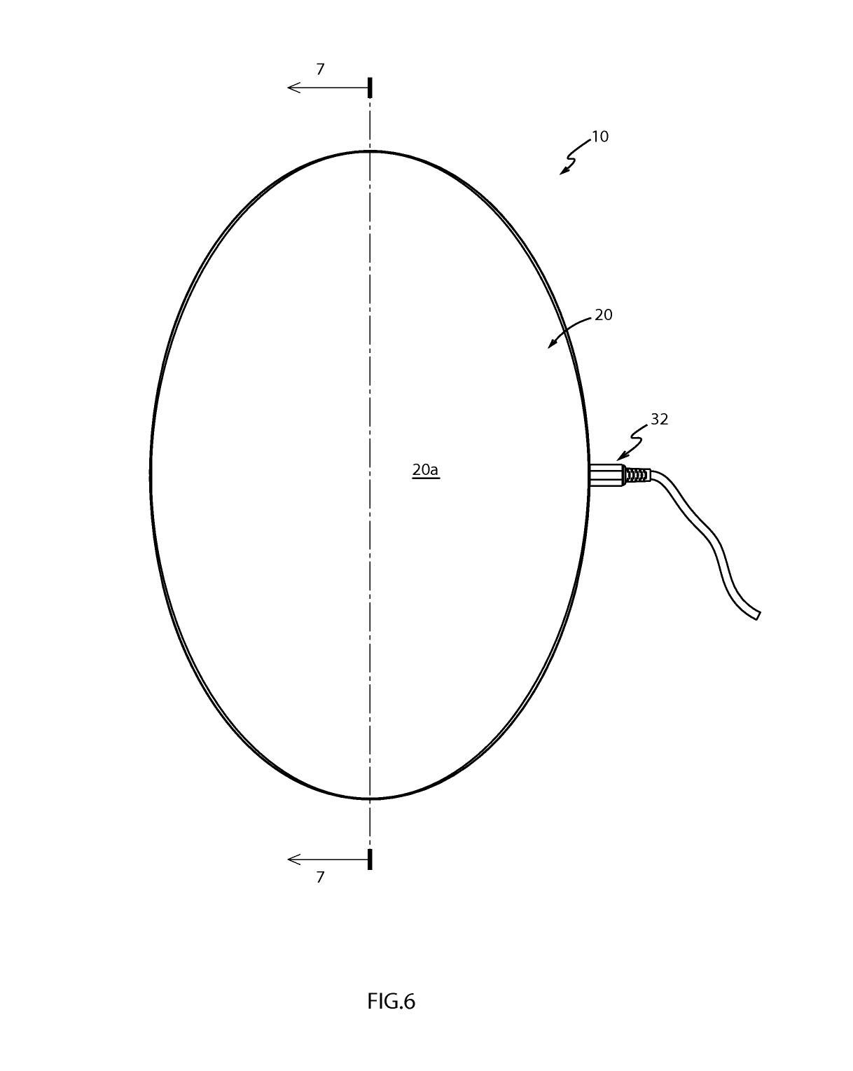

Top cover 20 is ergonomically configured so that it may be easily held in the palm of the user's hand. In particular, top cover 20 is generally elliptical in shape when viewed from the top (FIG. 6) and is convex when viewed from the end (FIG. 3) or side (FIG. 4). Top cover 20 has a wall 20a contoured to be receivable in a user's palm and thus includes a raised central region and sloping sides and ends. Top cover's sides and ends terminate in a bottom rim 20b. Top cover 20 is substantially hollow and defines an interior chamber 20c.

First insulation layer 22 has a surface 22a that is configured to be substantially complementary to the interior surface of wall 20a of top cover 20 and first insulation layer 22 is nestingly received in interior chamber 20c of top cover 20. Surface 22a of first insulation layer 22 abuts the interior surface of wall 20a. First insulation layer 22 terminates in a bottom rim 22b. Bottom surface of first insulation layer 22 defines a cavity 22c therein.

Cooling pad 16 has a wall 16a that is configured substantially complementary to the bottom surface of first insulation layer 22 and cooling pad 16 is nestingly received within cavity 22c of first insulation layer 22. First insulation layer 22 thus surrounds all but a portion of a bottom wall 16b of cooling pad 16 and thereby substantially prevents cold from escaping from cooling pad 16 into the environment other than through bottom wall 16b. Bottom wall 16b preferably is slightly convex or generally horizontal except for a region adjacent an outer edge 16c thereof.

Retainer 24 has an upper end 24a and a lower end 24b. Upper end 24a is configured to engage bottom edge 20b of top cover 20. In particular, a flange 24c extends upwardly and outwardly from upper rim 24a to interlock with bottom edge 20b. Lower end 24b of retainer 24 is configured to circumscribe and engage an outer rim 16c of bottom wall 16b of cooling pad 16 and to keep cooling pad 16 locked within cavity 20c of top cover 20. Lower rim 24b of retainer 24 defines an aperture 24d therein and through which a portion of bottom wall 16b of cooling pad 16 is accessible. It is this portion of bottom wall 16b that is brought into contact with the user's skin when the device 10 is used to stop a hot flash.

As indicated previously, cradle 14 includes bottom cover 26. Bottom cover 26 has an upper rim 26a (FIG. 9) that is configured to be complementary to a bottom region of hand-held assembly 12. That bottom region of handheld assembly 12 includes a lower surface of retainer 24 and the portion of the bottom wall 16b of cooling pad 16 which will be brought into contact with the user's skin. As shown in FIG. 3, retainer 24 preferably is slightly larger in external dimensions than is bottom cover 26 so that a lip 12a of hand-held assembly 12 projects outwardly beyond a side wall 14a of cradle 12. Lip 12a makes it easier for the user to grasp assembly 12 and remove it from cradle 14.

Bottom cover 26 of cradle 14 defines a recessed region 26b (FIG. 9) therein that is configured to receive contact pad 34 therein. Contact pad 34 is secured to bottom cover 26 in any suitable manner such as by way of fasteners 40. Bottom cover 26 defines a pair of apertures 26c therein, where the apertures 26c are located in recessed region 26b. Bottom cover 26 further includes a pair of flanges 26d that are generally semi-circular in shape and flattened. Flanges 26d extend downwardly from upper surface 26a of bottom cover 26.

Contact pad 34 has a top surface 34a that is complementary to bottom wall 16b of cooling pad 16 and a bottom surface 34b that is disposed adjacent an upper surface of bottom cover 26 in recessed region 26b. When hand-held assembly 12 is docked in cradle 14, top surface 34a of contact pad 34 is disposed in abutting contact with bottom wall 16b of cooling pad 16 and thus thermal transfer between contact pad 34 and cooling pad 16 is facilitated.

A Peltier-effect element 36 is engaged in each aperture 26c of bottom cover 26 and extends for a distance outwardly therefrom. An upper surface 36a of each element 36 is disposed in thermal contact with bottom surface 34b of contact pad 34. (As is known in the art, thermal grease may also be applied to the upper and lower surface of elements 36.) The Peltier-effect elements 36 are provided as solid state heat pumps that are used to lower the temperature of the contact pad 34 to around 10 degrees Celsius. This is about ten degrees lower than a normal ambient temperature of 24 degrees Celsius. Device 10 may also be provided with a microprocessor (not shown) that is able to be programmed to cause the Peltier-effect elements 36 to cool contact pad 34 to temperatures lower or higher than 10 degrees Celsius if this was desirable or necessary for the device to function in the manner contemplated. The temperature of contact pad 34 needs to be sufficiently low enough to stimulate the cold thermoreceptors in the skin at the back of the user's neck to send a signal to the user's hypothalamus to stop the cold flash.

When the Peltier-effect elements 36 are charged, as will be hereinafter described, the upper surface 36a thereof will be rapidly cooled and will thereby cool contact pad 34.

Second insulation layer 28 is configured to be substantially complementary to lower surface of bottom cover 26 and is engaged therewith. Second insulation layer 28 has an upper end 28a and a recessed region 28b with one or more apertures 28c defined therein. Apertures 28c are substantially shaped and located to align with apertures 26b of bottom cover 26. Second insulation layer 28 also includes two flattened zones 28c that are shaped and located to align with flanges 26d on bottom cover 26 when second insulation layer 28 is disposed around bottom cover 26. When second insulation layer 28 is engaged with bottom cover 26, the Peltier-effect elements 36 are received through the aligned apertures 26b, 28c as is shown in FIG. 7.

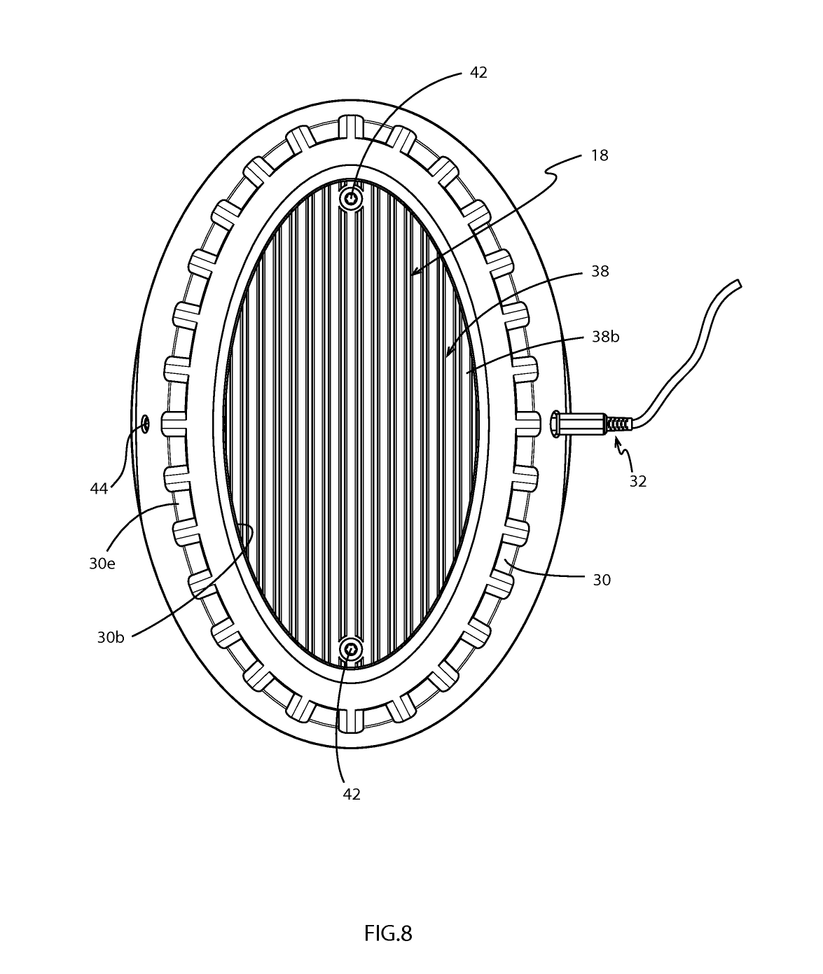

Heat sink 38 has a generally flat upper surface 38a that is secured adjacent a bottom surface of recessed region 28b of second insulation layer 28 by way of fasteners 42. Upper surface 38a of heat sink 38 is in contact with a bottom surface of each Peltier-effect element 36. As best seen in FIG. 8, bottom surface 38b of heat sink 38 includes a plurality of ridges and grooves. As will be described further herein, heat sink 38 absorbs heat emitted by the Peltier-effect elements 36 as they rapidly cool contact pad 34. The alternating ridges and grooves in heat sink 38 provide additional surface area for heat sink 38 to dissipate heat into the surrounding environment.

Housing 30 has a wall 30a configured to circumscribe and surround the exterior surface of second insulation layer 38 and to interlock with outer rim of bottom cover 26. Housing 30 defines an opening 30b therein and through which the bottom surface 30b of heat sink 38 is accessible. Housing 30 also is provided with a first aperture 30c through which the AC-DC adapter 32 is connected, and is provided with a second aperture 30d through which a light emitting diode (LED) 44 is visible. Although it is not illustrated herein it should be understood that cradle 14 includes electric circuitry which operatively connects AC-DC adapter 32, Peltier-effect elements 36 and LEDs 44. The AC-DC adapter 32 is connected to a remote power source such as a wall outlet (not shown). A bottom edge 30e of housing 30 is configured into a plurality of alternating ridges and gaps, the purpose of which will be described hereafter.

If provided, the microprocessor which controls device 10 is programmed to include various thermal protection override features and safety features. The microprocessor monitors and automatically shuts off device 10 if any operational issues arise. The electrical circuitry that connects the various components within cradle 14 is not shown for the sake of clarity.

In use, device 10 is plugged into a source of AC power by way of AC-DC adapter 32. LED 32 glows to indicate that the device 10 is powered. Power is applied via adapter 32 to Peltier-effect elements 36 which then become rapidly cooled on their upper surfaces 36a. The cold is conducted into lower surface 34b of contact pad 34, thereby cooling the same. At the same heat generated by devices 36 is conducted into upper surface 38a of heat sink 38 and is subsequently released into the surrounding environment through the gaps in bottom end 30e of housing 30. Heat may also be released into a surface (not shown) upon which housing 30 rests.

The cold conducted from Peltier-effect elements 36 into contact pad 34 is, in turn, conducted into cooling pad 16 when hand-held assembly 12 is engaged in cradle 14. Because of the presence of the first insulation layer 22, the cold in cooling pad 16 is retained therein and is not dissipated into the surrounding air. (Because of the presence of the second insulation layer 28, the heat transferred to heat sink 38 is not transmitted upwardly into contact pad 34.) Since Peltier-effect elements 36 are constantly powered because device 10 is plugged in, contact pad 34 and therefore cooling pad 16 are always sufficiently cold enough for use to stop a hot flash at the first sign of the same. Additionally, if hand-held assembly 12 is removed from cradle 14 and is then returned thereto, the cooling pad 16 is able to be rapidly recharged with cold by coming into contact with contact pad 34 and is therefore available for use in a matter of seconds.

The flanges 26d on bottom cover 26 enable a user to quickly and easily locate a suitable region on device 10 at which hand-held assembly 12 may be easily grasped as they provide areas of easier access to engage lip 12a on assembly 12, even in the dark.

Device 10 is used in the following manner on the skin on the back of the neck of a woman at the first sign of the onset of a hot flash. The woman will lift hand-held assembly 12 off cradle 14 and, since cradle 14 is permanently plugged into an AC power source, the hand-held assembly 12 is immediately ready for use. Bottom surface 16b of cooling pad 16 is placed in contact with the skin at the back of the user's neck. Because cooling pad 16 is cold, the skin is cooled, thereby causing the thermoreceptors in the skin to be stimulated to a degree sufficient to emit a signal to the nervous system. Device 10 is held in a first position until the cold sensation on the neck begins to subside. This subsidence of the sensation occurs as the thermoreceptors are stimulated to a maximum degree, a condition known as saturation. Once saturation is reached, the thermoreceptors will no longer emit a signal to the brain. In order to prevent the cessation of a signal to the brain, the woman must move device 10 on her skin from the first position to a second position spaced a distance from the first position. The thermoreceptors in the skin at the second position are stimulated and thus transmit a signal to the hypothalamus. The thermoreceptors in the first area of the skin will reset themselves after a time period of about five to ten seconds. This means that after a five to ten second period, the thermoreceptors are once again in a condition where they may be stimulated and will once again emit a signal to the brain. The woman will move device back and forth between at least the first and second positions to keep the hypothalamus stimulated. The woman keeps “shuttling” the hand-held assembly 12 back and forth in this manner, holding the device in each new position until the cold sensation of the plates on the skin begins to subside, before she changes the device's position on the back of the neck. The woman will typically have to relocate assembly 12 every ten to fifteen seconds in order to have cooled plate 16 contact “new” skin where the skin's thermoreceptors have not been stimulated or have already been reset. This shuttling motion helps to keep the thermoreceptors in the skin firing signals to the hypothalamus at a maximum rate. After a time period of around sixty seconds, assembly 12 may be removed from further contact with the skin 12. This time period of activation has been found to be sufficient to stop the symptoms of a hot flash before they are fully expressed. Hand-held assembly 12 is then repositioned on cradle 14.

It will be understood that while the device has been described specifically for reducing the effects of menopausal hot flashes, the device could be used as a heat massage device for the neck. Peltier-effect elements are constructed in such a manner that one surface, such as surface 36a, becomes cooled and the opposite surface becomes heated. The determination as to which of the two surfaces is heated or cooled results from the polarity across the device 36. So, for example, if one wished to heat plate 16 instead of cooling the same, the DC polarity across the Peltier-effect element 36 could be reversed. Plate 16 would then become cooled and heat sink 38 would become heated. Plate 16 could then be brought in contact with the skin to heat and/or massage the same.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of the device is an example and the device is not limited to the exact details shown or described.

1. A device for the non-invasive treatment of menopausal hot flashes comprising:
a hand-held assembly sized to be held in the palm of a user's hand;
a cooling plate disposed within the hand-held assembly and being adapted to be placed in contact with a region of a user's skin to cool the same at the onset of a hot flash;
a docking cradle configured to receive the hand-held assembly and to lower the temperature of the cooling plate disposed therein; and wherein the cradle keeps the docked cooling plate at the lowered temperature and therefore ready for use.

2. The device as defined in claim 1, further comprising an AC-DC adapter engaged with the cradle and being adapted to be operatively connected to a remote power source.

3. The device as defined in claim 1, where the cradle includes:
a cooling system configured to be operatively connected to the cooling plate in the hand-held assembly when the assembly is docked with the cradle.

4. The device as defined in claim 3, wherein the cooling system is a solid state cooling system.

5. The device as defined in claim 2, wherein the cooling plate comprises a portion of a lowermost surface of the handheld assembly and the cooling system comprises a portion of an uppermost surface of the cradle; and the cooling plate and cooling system abut each other when the assembly is docked in the cradle.

6. The device as defined in claim 5; wherein the cooling system includes:
a contact plate positioned to abut the cooling plate of the assembly; and
a Peltier-effect element having a first surface abutting the contact plate; said Peltier-effect element being adapted to be operatively connected via the AC-DC adapter to the remote power source and to remain in a charged condition when so connected.

7. The device as defined in claim 6, further comprising a heat sink disposed to abut a second surface of the Peltier-effect element.

8. The device as defined in claim 1, further comprising a first insulation layer provided in the hand-held assembly to surround all but a lower surface of the cooling plate.

9. The device as defined in claim 7, further comprising a second insulation layer provided in the cradle, said second insulation layer being positioned to isolate the contact plate from the heat sink and thereby prevent thermal transfer between the contact plate and heat sink.

10. A method of controlling menopausal hot flashes in a woman's body comprising the steps of:
a) activating a therapeutic device in a cradle to cool a thermally conductive cooled plate in a hand-held assembly of the device;
b) lifting the hand-held assembly from the cradle at a first sign of a hot flash;
c) bringing the cooled plate into abutting contact with a first region of skin at the back of the woman's neck.

11. The method as defined in claim 10, further comprising the steps of:
d) holding the cooled plate in contact with the first region of skin for a first period of time; and
e) physically moving the hand-held assembly along the skin at the back of the woman's neck until the cooled plate contacts a second region of skin at the back of the woman's neck remote from the first region of skin; and
f) holding the cooled plate in contact with the second region of skin for a second period of time.

12. The method as defined in claim 11, further comprising the steps of:
g) moving the handheld assembly along the skin until the cooled plate contacts one of a third region of skin at the back of the neck and the first region of skin; and
h) holding the cooled plate in contact with the one of the first and third regions of skin for a third period of time.

13. The method as defined in claim 12, further comprising the steps of repeating steps c) through h) until the hot flash ceases.

14. The method as defined in claim 13 wherein each of the first and second periods of time are between ten and fifteen seconds long.

15. The method as defined in claim 14, wherein the third period of time is between ten and fifteen seconds long.

16. The method as defined in claim 13 further comprising the step of returning the hand-held assembly to the cradle.

17. The method as defined in claim 13, further comprising the step of keeping the cradle plugged into an AC power source.

18. The method as defined in claim 17 further comprising the step of keeping the cooled plate at a temperature of about 10 degrees Celsius when the hand-held assembly is engaged with the cradle.

19. A therapeutic device comprising:
a hand-held assembly containing a first thermally conductive plate;
a cradle configured to dock with the hand-held assembly; said cradle being adapted to be engaged with a remote power source;
a heating and cooling system disposed within the cradle;
a second thermally conductive plate disposed within the cradle and thermally engaged with the heating and cooling system to be heated or cooled thereby; and wherein the first and second thermally conductive plates are brought into abutting contact with each other when the hand-held assembly is engaged with the cradle and after the assembly is undocked from the cradle, the first thermally conductive plate is brought into contact with a person's skin to impart heating or cooling to the same.

20. The device as defined in claim 19, wherein the heating and cooling system includes a Peltier-effect element that is thermally connected to the second conductive plate and a heat sink that is thermally connected to the Peltier-effect element.
